(19) United States
(12) Patent Application Publication
(10) Pub. No.: US 2010/0240121 A1
(43) Pub. Date: Sep. 23, 2010

(54) Electrospun nanofibrous membrane assembly for use in capturing chemical and/or biological analytes

(76) Inventors: Kris J. Senecal, North Smithfield, RI (US); Andre G. Senecal, North Smithfield, RI (US); Philip E. Pivarnik, Narragansett, RI (US); Charlene M. Mello, Rochester, MA (US); Jason W. Soares, Shrewsbury, MA (US); Heidi L. Schreuder-Gibson, Holliston, MA (US)

Correspondence Address: U.S. ARMY SOLDIER SYSTEMS CENTER;LEGAL OFFICE, A306, 15 KANSAS STREET, NATICK, MA, 01760-5035, US

(21) Appl. No.: 11/388,175

(22) Filed: Mar. 24, 2006

Related U.S. Application Data
(62) Division of application No. 11/029,548, filed on Jan. 5, 2005.

(51) Int. Cl. C12M 1/34

(52) U.S. Cl. 435/287.2; 435/287.9

(57) ABSTRACT

A membrane assembly adapted for use in capturing an analyte of interest, the membrane assembly comprising in one embodiment (a) an electrospun nanofibrous membrane, the electrospun nanofibrous membrane comprising a random mat of electrospun nanofibers, at least some of the electrospun nanofibers including one or more types of functional groups; and (b) at least one molecular recognition element immobilized on the random mat via a functional group, the molecular recognition element being adapted to selectively bind the analyte of interest. The membrane assembly may be incorporated into a sensor.

### STATEMENT REGARDING FEDERALLY SPONSORED RESEARCH OR DEVELOPMENT

The invention described herein may be manufactured and used by or for the government of the United States of America for governmental purposes without the payment of any royalties thereon or therefor.

### BACKGROUND OF THE INVENTION

The present invention relates generally to implements for use in capturing and/or detecting chemical and/or biological analytes and relates more particularly to a novel implement for use in capturing and/or detecting chemical and/or biological analytes.

Microorganisms, such as bacteria, viruses, fungi and protozoa, are commonplace in the environment. Although many such microorganisms are innocuous to humans, certain species of microorganisms are pathogenic and pose a serious health risk to people. Exposure to such pathogenic microorganisms may be inadvertent, such as in the case of poorly handled or poorly prepared foods containing Salmonella, E. coli 0157:H7 or the like, or may be deliberate, such as in the case of biological weapons armed with spores of anthrax or the like. As can readily be appreciated, in view of the above, it would be highly desirable to be able to remove pathogenic microorganisms, as well as natural or synthetic toxins and other deleterious chemicals, from various media, such as food, water and air, that are likely to come into human contact and/or to detect the presence of such materials in these media. Unfortunately, the presence of many undesirable chemical or biological materials cannot typically be ascertained simply by visual or other sensory examination of a sample, but rather, requires the use of specialized testing equipment and procedures. Moreover, because certain chemical or biological materials may be lethal in very small doses (for example, certain pathogenic microorganisms in doses constituting as few as about ten microorganisms), the ability to remove and/or to detect minute quantities of such materials is highly desirable.

One technique that is commonly used to detect the presence of a pathogenic microorganism or a toxin within a sample is an enzyme linked immunosorbent assay (ELISA), such a technique employing, among other things, an antibody adapted to bind to the microorganism or toxin of interest. Examples of ELISA techniques used in the detection of these types of deleterious substances may be found in the following U.S. patents, all of which are incorporated herein by reference: U.S. Pat. No. 6,174,667, inventors Huchzermeier et al., which issued Jan. 16, 2001; U.S. Pat. No. 6,124,105, inventors Verschoor et al., which issued Sep. 26, 2000; U.S. Pat. No. 5,294,537, inventor Batt, which issued Mar. 15, 1994; and U.S. Pat. No. 4,486,530, inventors David et al., which issued Dec. 4, 1984.

Another technique that is commonly used to detect the presence of a pathogenic microorganism within a sample is a DNA-based approach that involves detecting within the sample the presence of one or more genes indicative of the pathogenic microorganism of interest. Such a DNA-based approach typically comprises inoculating a culture broth with a sample under investigation, allowing the broth to culture for a period of time (e.g., typically overnight up to a few days), isolating any microorganisms present in the broth, retrieving the DNA from the isolated microorganisms, amplifying the retrieved DNA, and using one or more hybridizing probes specific for a gene or genes of interest to detect the presence of said gene(s) within the amplified DNA.

Examples of other techniques for detecting the presence of a pathogenic microorganism in a sample are disclosed in U.S. Pat. No. 6,159,719, inventors Laine et al., which issued Dec. 12, 2000; U.S. Pat. No. 5,750,357, inventors Olstein et al., which issued May 12, 1998; Pyle et al., “Sensitive Detection of Escherichia coli O157:H7 in Food and Water by Immunomagnetic Separation and Solid-Phase Laser Cytometry,” Appl. Environ. Microbiol., 65(5):1966-72 (May 1999) and Sharma et al., “Semi-automated fluorogenic PCR assays (TaqMan) for rapid detection of Escherichia coli O157:H7 and other Shiga toxigenic E. coli,” Molecular and Cellular Probes, 13:291-302 (1999), all of which are incorporated herein by reference.

Typically, the antibody, peptide or other molecular recognition element used to bind to the microorganism or other analyte of interest is immobilized in some fashion on a substrate. For example, the molecular recognition element may be immobilized via a chemical a chemical linker to polystyrene beads, to the wells of a microtiter plate, or to a polymeric film. As can readily be appreciated, the sensitivity of a capturing/detection technique is dependent, to a considerable extent, upon the number of molecular recognition elements to which the sample is exposed. In other words, the greater the number of available molecular recognition elements, the greater the likelihood that the analyte of interest will be captured/detected. Where, however, the molecular recognition elements are immobilized in the conventional manner on the outer surfaces of beads, along the walls of the wells of a microtiter plate or the like, it should be recognized that there is an upper limit to the number of molecular recognition elements that may be immobilized in any given area.

Nanofibrous membranes made by electrospinning have attracted some attention over the last several years as being useful as wound dressings and HEPA (High Efficiency Particulate Arrestance) filters. Electrospinning is an atomization process of a conducting fluid which exploits the interactions between an electrostatic field and the conducting fluid. When an external electrostatic field is applied to a conducting fluid (e.g., a semi-dilute polymer solution or a polymer melt), a suspended conical droplet is formed, whereby the surface tension of the droplet is in equilibrium with the electric field. Electrostatic atomization occurs when the electrostatic field is strong enough to overcome the surface tension of the liquid. The liquid droplet then becomes unstable and a tiny jet is ejected from the surface of the droplet. As it reaches a grounded target, the material can be collected as an interconnected web containing fine, i.e., nanosize diameter, fibers. The resulting membranes from these nanofibers have very large surface area to volume ratios and small pore sizes. Additional background information regarding electrospun nanofibrous membranes may be found, for example, in U.S. Pat. No. 4,323,525, inventor Bornat, which issued Apr. 6, 1982; U.S. Pat. No. 4,143,196, inventors Simm et al., which issued Mar. 6, 1979; and U.S. Pat. No. 4,043,331, inventors Martin et al., issued Aug. 23, 1977, all of which are incorporated herein by reference.

Recently, electrospun nanofibrous membranes have garnered attention for other possible applications. For example, in U.S. Pat. No. 6,800,155, inventors Senecal et al., which issued Oct. 5, 2004, and which is incorporated herein by reference, there is disclosed a conductive (electrical, ionic, and photoelectric) polymer membrane article that may be used, for example, in textiles, the article comprising a non-woven membrane of polymer fibers, wherein at least some of the fibers have diameters of less than one micron and wherein the membrane has an electrical conductivity of at least about 10⁻⁶ S/cm. Also disclosed in the aforementioned patent is a method of making such an article, said method comprising electrostatically spinning a spin dope comprising a polymer carrier and/or a conductive polymer or conductive nanoparticles to provide inherent conductivity in the article.

In U.S. Pat. No. 6,558,422, inventors Baker et al., which issued May 6, 2003, and which is incorporated herein by reference, indented structures are disclosed that each include (a) a body defining a plurality of indentations, substantially all of the plurality of indentations including a surface layer including a biologically active substance; and (b) a body surface, wherein each of the plurality of indentations opens onto the body surface through a plurality of openings, and wherein the biologically active substance is not substantially present on the body surface. Examples of such structures include medical devices, such as medical devices that are completely or partially implantable into a living body. The surface layer of the indentations (or at least some of the indentations) of the medical devices may include biologically active molecules, such as proteins, that promote the growth of cells into and/or within the indentations, thereby promoting the acceptance of the implanted device by the living body.

In U.S. Patent Application Publication No. US 2002/0173213 A 1, inventors Chu et al., which was published Nov. 21, 2002, and which is incorporated herein by reference, there are disclosed biodegradable and/or bioabsorbable fibrous articles and methods for using the articles in medical applications. The biodegradable and/or bioabsorbable fibrous articles, which are formed by electrospinning fibers of biodegradable and/or bioabsorbable fiberizable material, comprise a composite (or asymmetric composite) of different biodegradable and/or bioabsorbable fibers. Articles having specific medical uses include an adhesion-reducing barrier and a controlled delivery system. The methods include methods for reducing surgical adhesions, controlled delivery of a medicinal agent and providing controlled tissue healing.

In U.S. Patent Application Publication No. US 2002/0081732 A1, inventors Bowlin et al., which was published Jun. 27, 2002, and which is incorporated herein by reference, there are disclosed compositions that comprise an electroprocessed material and a substance, the substance being releasably coupled to the electroprocessed material. The electroprocessed material may be one or more natural materials, one or more synthetic materials or a combination thereof. The substance may be one or more therapeutic or cosmetic substances or other compounds, molecules, cells, or vesicles. The compositions can be used in substance delivery, including drug delivery within an organism by, for example, releasing substances or containing cells that release substances. Although the aforementioned published patent application makes a passing reference in paragraph [0179] thereof to an embodiment in which antibodies are immobilized on an electroprocessed matrix, no specific teaching is provided as to exactly how such antibodies may be immobilized on the electroprocessed matrix. Moreover, with respect to such an embodiment, the aforementioned published patent application only discloses using such an embodiment to bind a desired molecule and does not teach or suggest using the subject composition to detect desired molecules.

Other patents that may be of interest include the following, all of which are incorporated herein by reference: U.S. Patent Application Publication No. US 2003/0100944 A1, inventors Laksin et al., which was published May 29, 2003; U.S. Patent Application Publication No. US 2003/0065355 A1, inventor Weber, which was published Apr. 3, 2003; U.S. Patent Application Publication No. US 2003/0021821 A1, inventors Fertala et al., which was published Jan. 30, 2003; U.S. Patent Application Publication No. US 2002/0124953 A1, inventors Sennett et al., which was published Sep. 12, 2002; U.S. Patent Application Publication No. US 2002/0122840 A1, inventors Lee et al., which was published Sep. 5, 2002; U.S. Patent Application Publication No. US 2002/0096246 A1, inventors Sennett et al., which was published Jul. 25, 2002; U.S. Pat. No. 6,306,424, inventors Vyakarnam et al., which issued Oct. 23, 2001; U.S. Pat. No. 6,306,419, inventors Vachon et al., which issued Oct. 23, 2001; U.S. Pat. No. 6,110,590, inventors Zarkoob et al., which issued Aug. 29, 2000; U.S. Pat. No. 6,106,913, inventors Scardino et al., which issued Aug. 22, 2000; and U.S. Pat. No. 5,840,387, inventors Berlowitz-Tarrant et al., which issued Nov. 24, 1998.

### SUMMARY OF THE INVENTION

It is an object of the present invention to provide a novel implement for use in capturing and/or detecting chemical and/or biological analytes of interest.

It is another object of the present invention to provide an implement as described above that overcomes at least some of the shortcomings discussed above in connection with existing implements.

Therefore, in accordance with the teachings of the present invention, there is provided a membrane assembly adapted for use in capturing an analyte of interest; said membrane assembly comprising in one embodiment (a) an electrospun nanofibrous membrane, said electrospun nanofibrous membrane comprising a random mat of electrospun nanofibers, at least some of said electrospun nanofibers including one or more types of functional groups; and (b) at least one type of molecular recognition element covalently bonded to at least one of said one or more types of functional groups, said molecular recognition element being adapted to selectively bind the analyte of interest.

In the above embodiment, the electrospun nanofibers that include one or more types of functional groups may comprise a blend or mixture of different types of polymers, at least some of which possess functional groups, or may consist of a single type of polymer that possesses a functional group. For example, at least some of the electrospun nanofibers may comprise a blend or mixture of polyurethane and polyamine or may consist of a carboxylated polyvinyl chloride. In addition, the electrospun nanofibers may further include one or more types of molecular recognition elements incorporated into the electrospun nanofibers, themselves.

In another embodiment, there is provided a membrane assembly adapted for use in capturing an analyte of interest, said membrane assembly comprising (a) an electrospun nanofibrous membrane; and (b) at least one type of molecular recognition element non-covalently associated with said electrospun nanofibrous membrane, said at least one type of molecular recognition element being adapted to selectively bind the analyte of interest.

Preferably, the aforementioned electrospun nanofibrous membrane comprises a mat (e.g., ordered, patterned, random or otherwise) of electrospun nanofibers, at least some of said electrospun nanofibers including one or more avidin molecules, and at least some of said at least one type of molecular recognition element is biotinylated and immobilized on said mat via said one or more avidin molecules.

The present invention is also directed to a membrane adapted for use in capturing an analyte of interest, said membrane being formed by a method comprising the steps of (a) providing a spin dope; (b) adding at least one type of molecular recognition element to the spin dope, the molecular recognition element being adapted to selectively bind the analyte of interest; and (c) then, using the spin dope produced in step (b) to form an electrospun nanofibrous membrane.

The present invention is also directed to methods of detecting analytes using the above-described membrane assemblies and to sensors including the above-described membrane assemblies.

Additional objects, as well as features and advantages, of the present invention will be set forth in part in the description which follows, and in part will be obvious from the description or may be learned by practice of the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### DETAILED DESCRIPTION OF PREFERRED EMBODIMENTS

As noted above, the present invention is directed at a membrane assembly adapted for use in capturing an analyte of interest, such as, but not limited to, a pathogenic or non-pathogenic microorganism, a synthetic or natural toxin, or a contaminant or impurity in a sample. The membrane assembly of the present invention comprises (a) an electrospun nanofibrous membrane, said electrospun nanofibrous membrane comprising a non-woven or random mat of electrospun nanofibers; and (b) at least one type of molecular recognition element immobilized on said non-woven or random mat, said molecular recognition element being adapted to selectively bind the analyte of interest. As can be appreciated, because an electrospun nanofibrous membrane has a high surface area to volume ratio, molecular recognition elements may be immobilized on the membrane in large numbers, thereby making the assembly highly sensitive for the analyte in question.

According to a first embodiment, the electrospun nanofibrous membrane is prepared by electrospinning together two or more different types of polymers to produce blended/mixed nanofibers arranged in a non-woven or random mat, at least some of the co-electrospun polymers possessing one or more functional groups to which the molecular recognition element is covalently bonded. Examples of suitable functional groups that may be present in the blended/mixed nanofibers include, but are not limited to, hydroxyl, aldehyde, carbonyl, carboxyl, amino, amido, thiol, ester, ether, and disulfide groups. For example, the membrane may be prepared by co-electro spinning polyurethane and polyamine to yield a non-woven membrane having blended/mixed nanofibers with amine functional groups. Other types of blended/mixed nanofibers may be obtained by co-electrospinning one or more types of proteins with one or more non-proteinaceous polymers.

It should be appreciated that one could electrospin together a plurality of different types of polymers to form a blended/mixed nanofiber having two or more different types of functional groups. In this manner, different types of functional groups could be used to covalently bond different types of molecular recognition elements. (This may desirable in those instances in which certain molecular recognition elements are chemically compatible for covalent bonding with certain types of functional groups but not with other types of functional groups.)

Conductive polymers and/or conductive nanoparticles may additionally be incorporated into the blended/mixed nanofibers to endow the membrane with desirable electric, ionic and/or photoelectric properties. Examples of suitable conductive polymers include, but are not limited to; polyaniline, polypyrrole, polythiophene, polyphenol, polyacetylene, and polyphenylene. Examples of suitable conductive nanoparticles include titanium dioxide, zinc oxide; tin sulfide and tin oxide.

Molecular recognition elements (e.g., in powder form or in solution) may also be added to the spin dope prior to fiber formation as a way to incorporate the molecular recognition elements into the fibers, themselves.

According to a second embodiment, the electrospun nanofibrous membrane is prepared by electrospinning a single type of polymer, said single type of polymer itself possessing functional groups. An example of a polymer suitable for this purpose is a carboxylated polyvinyl chloride polymer.

According to a third embodiment, the molecular recognition elements are biotinylated and the electrospun fibers include avidin for non-covalently binding the biotinylated molecular recognition elements.

The molecular recognition elements suitable for use in the membrane assembly of the present invention include, but are not limited to, antibodies, aptamers, peptides, PNAs (peptide nucleic acids) and liposaccharides. Antibodies that specifically bind enzymes or fluorescent compounds, such as glucose oxidase, horseradish peroxidase, or fluoescein can be selectively immobilized onto the nanoporous membranes and evaluated for their pre-concentration capabilities. These antibodies do not require secondary labeling and can therefore be used to directly quantify the antibody attachment and activity in association with the electrospinning process.

Aptamers are artificial receptors comprising DNA oligomers that are designed for the specific binding of target analytes. Aptamers offer a potential advantage over antibodies in that they eliminate the need for an animal host for their production. Also, aptamers offer a completely in vitro combinatorial chemistry alternative to traditional protein-based antibody technology.

Examples of suitable peptides for use as molecular recognition elements include antimicrobial peptides, examples of which are disclosed in the following documents, all of which are incorporated herein by reference: U.S. Pat. No. 6,042,848, inventors Lawyer et al., which issued Mar. 28, 2000; U.S. Pat. No. 5,914,248, inventors Kuipers et al., which issued Jun. 22, 1999; U.S. Pat. No. 5,912,230, inventors Oppenheim et al., which issued Jun. 15, 1999; U.S. Pat. No. 5,889,148, inventors Lee et al., which issued Mar. 30, 1999; U.S. Pat. No. 5,885,965, inventors Oppenheim et al., which issued Mar. 23, 1999; U.S. Pat. No. 5,861,275, inventor Hansen, which issued Jan. 19, 1999; U.S. Pat. No. 5,856,127, inventors Powell et al., which issued Jan. 5, 1999; U.S. Pat. No. 5,844,072, inventors Selsted et al., which issued Dec. 1, 1998; U.S. Pat. No. 5,798,336, inventors Travis et al., which issued Aug. 25, 1998; U.S. Pat. No. 5,646,119, inventors Oppenheim et al., which issued Jul. 8, 1997; U.S. Pat. No. 5,631,228, inventors Oppenheim et al., which issued May 20, 1997; U.S. Pat. No. 5,519,115, inventors Mapelli et al., issued May 21, 1996; U.S. Pat. No. 5,447,914, inventors Travis et al., which issued Sep. 5, 1995; Epand et al., “Diversity of antimicrobial peptides and their mechanisms of action,” Biochimica et Biophysica Acta, 1462:11-28 (1999); Nicolas et al., “Peptides as Weapons Against Microorganisms in the Chemical Defense System of Vertebrates,” Annu. Rev. Microbiol., 49:277-304 (1995); Moore et al., “Antimicrobial peptides in the stomach of Xenopus laevis,” J. Biol. Chem., 266:19851-7 (1991); and Lee et al., “Antibacterial peptides from pig intestine: isolation of a mammalian cecropin,” Proc. Natl. Acad. Sci. U.S.A., 86:9159-62 (1989). Specific antimicrobial peptides that may be suitable for use in the present invention include Cecropin P1, Indolicidin and PGQ antimicrobial peptides.

As can be appreciated, analytes captured using the membrane assembly of the present invention can be detected, for example, using another molecular recognition element directed against the analyte that is conjugated to a fluorescent label or to a peroxidase (for colorimetric analysis).

The following examples are provided for illustrative purposes only and are in no way intended to limit the scope of the present invention:

### Example 1

### Electrospinning Procedure Employed

To electrospin the polymers, a variable high voltage power supply purchased from Gamma High Voltage Research (Ormond Beach, Fla.) was used. A glass pipette used in the spinning process was tilted from horizontal so that a small drop of polymer solution was maintained at the capillary tip due to the surface tension of the solution. The electrospinning apparatus also included a stainless steel mesh screen placed 10-15 cm horizontally from the tip of the pipette as the grounded counter electrode. The potential difference between the pipette and the counter electrode used to electrospin the polymer solution was 10-15 KV. Fiber diameters of the polymeric nanofibers ranged from 50-1000 nanometers depending on the percentage of polymer in solvent. The electrospinning process was performed at approximately 18,000 volts onto a stainless steel mesh wire screen for a total weight of polymer on the screen of 2 mg. After electrospinning, the screens were cut with a dye punch to uniform sizes to minimize size and weight variances.

### Example 2

### Membrane Assemblies Including Carboxylated PVC Membranes

A polymer solution comprising 6-10% w/w carboxylated polyvinyl chloride (Aldrich Chemical, St. Louis, Mo.) in an 85/15 solvent mixture of dimethyl formamide and tetrahydrofuran, respectively, was prepared: The carboxylated polyvinyl chloride polymer is said to have a 1.8% carboxyl-for-chloride substitution content. No further modification of the polymer was done prior to electrospinning in the manner provided in Example 1. The covalent linking of antibodies and peptides to the carboxylated polyvinyl chloride membrane was performed using either a combination of N-Hydroxysulfo-succinimide (NHS) (Pierce Chemical, Rockford, Ill.) and 1-ethyl-3-(3-Dimethyl aminopropyl)carbodiimide hydrochloride (EDC) (Pierce Chemical, Rockford, Ill.) or EDC alone. For purposes of the cross-linking reaction, the membrane in this case was treated like an initial protein with free carboxyl groups. Examples of antibodies that were covalently linked to carboxylated polyvinyl chloride membranes thus prepared were Goat anti-E. coli 0157:h7 and Goat anti-Rat/Rabbit anti-Goat HRP conjugated. Detection of the antibodies was determined using SUPER SIGNAL chemi-luminescence signal (Pierce Chemical, Rockford, Ill.) on a CHEMIGENIUS² chemiluminescence and fluorescence imaging system (SynGene, Frederick, Md.).

A peptide that was covalently linked to the carboxylated membrane was Nisin (Nisaplin Brand Nisin for Food stuff, aplin & Barrett, LTD, Trowbridge, Wilts., England) at an activity of 1,000,000 IU/gram. The Nisin was first solubilized and then covalently linked to the membrane using the above-identified NHS/EDC crosslinking reaction. In addition, Nisin was also incorporated into the membrane as a dried powder prior to electrospinning. The antimicrobial activity of this peptide while attached to the membrane was investigated by overlaying a lawn of bacteria (Micrococcus luteus) across the membrane and then checking overnight growth. “Zones of clearance” appearing as a cleared ring around the membranes indicated antimicrobial activity of the peptide.

### Example 3

### Membrane Assemblies Including Membranes with Amine Functional Groups

Electrospun membranes with primary amine functional groups were prepared by co-electrospinning two polymers, a water-soluble polyamine and a solvent-soluble polyurethane. A PAA-H-10C (poly(2-propen-1-amine, Nitto Boseki, LTD) solution in water was used as the amine fraction used to make the co-polymeric membrane. One hundred to seven hundred milligrams of PAA-H-10C solution were dried off in a vacuum to yield a more concentrated solution for inclusion in the spin dope prior to spinning. The polyurethane used was Pellethane 80AE (Dow Chemical, Midland, Mich.), which was solubilized 10% by weight in dimethyl formamide (DMF). The PAA-H-10C fraction was added to the polyurethane fraction with vigorous stirring at 60° C. Electrospinning was done quickly to avoid precipitation of either the polyamine or the polyurethane. Functional groups of the polyamine were then brought to the surface of the fibers by soaking the electrospun membrane in water overnight. The amine content of the co-spun membrane was determined using two systems: (i) Sulfo-SDTB (sulfo-succinimidyl-4-O-(4,4′-dimethoxytrityl)-butyrate) (Pierce Chemical, Rockford, Ill.) using the development system described in the Pierce literature and (ii) labeling with Fluorescein-EX (Molecular Probes, Eugene, Oreg.) detected with blue laser on the STORM 860 gel and blot imaging system (Molecular Dynamics, Sunnyvale, Calif.).

To covalently link peptides or antibodies to the above-described aminated electrospun membranes, the primary amines available on the surface of the membranes were treated using a hetero-bifunctional crosslinker, such as sulfo ECMS (Pierce Chemical, Rockford, Ill.). Detection of labeled antibodies or peptides linked to the electrospun membrane was performed using a TMB peroxidase system (KPL, Gaithersburg, Md.) or the STORM 860 gel and blot imaging system (Molecular Dynamics, Sunnyvale, Calif.) in both red and blue laser modes.

### Example 4

### Determination of Capture Efficiency

A number of carboxylated polyvinyl chloride membranes were prepared as in Example 2 above. Some of these membranes were then modified by covalent cross-linking to anti-E. coli 0157 KPL antibody while other of these membranes were not modified. Modified and unmodified membranes were then incubated with E. coli bacteria in relative concentrations of 10⁴, 10³, 10², and 10¹ and then were washed extensively with phosphate buffer. The capture efficiency was then determined by plating and growing the organisms captured by the membranes on MaConkey sorbital agar, specific for E. coli 0157. Results showed that the modified membranes were able to capture E. coli in all of the above concentrations. The concentration of 10¹ was determined to be 26 cells/ml based on plate cell growth studies done in parallel with the membrane bacterial cell capture study.

### Example 5

### Detection of Staph Entero Toxin B

Experiments were conducted to detect Staph Entero Toxin B (SEB) using electrospun carboxylated polyvinyl chloride membranes with attached anti-SEB antibodies to capture and concentrate the toxin. Highly purified SEB, rabbit anti-SEB biotinylated and HRP antibodies were covalently immobilized on the electrospun membrane and used in the SEB sandwich immunoassay study to detect the toxin. The test was designed to see if the electrospun membranes with antibodies specific for SEB could specifically capture and detect by chemiluminescence one of the SEB concentrations. It was interesting to learn that the membrane configuration could detect by a factor of greater than 2 the lowest 1 ng/ml concentration, demonstrating the potential sensitivity of the membrane capture system. It was demonstrated that the membranes could capture at the 1 ng/ml concentration of SEB Toxin.

The embodiments of the present invention recited herein are intended to be merely exemplary and those skilled in the art will be able to make numerous variations and modifications to it without departing from the spirit of the present invention. All such variations and modifications are intended to be within the scope of the present invention as defined by the claims appended hereto.

1. A sensor for use in capturing and detecting at least one analyte of interest, said sensor comprising:
(a) a membrane assembly having a nanofibrous membrane, said nanofibrous membrane comprising a non-woven, random mat of nanofibers, at least some of said nanofibers including one or more types of functional groups and said nanofibers including at least one type of molecular recognition element, said molecular recognition element being incorporated onto said nanofibers and being immobilized on said mat, said membrane having a surface area larger than a volume of said membrane, and said molecular recognition element being immobilized on the surface of the membrane in amounts large enough for making the assembly sensitive for the analyte, said analyte being pathogens in media for coming into human contact, said media comprising food, water, or air; and
(b) said at least one type of molecular recognition element covalently bonded to said random mat via at least one of said types of functional groups, said molecular recognition element for selectively binding the analyte of interest; wherein said functional group is selected from the group consisting of hydroxyl, aldehyde, carbonyl, carboxyl, amino, amido, thiol, ester, ether, and disulfide groups, and said molecular recognition element is selected from the group consisting of antibodies, aptamers, peptides, peptide nucleic acids and liposaccharides, and at least some of said electro spun nanofibers consist of a carboxylated polyvinyl chloride; and wherein at least some of said nanofibers comprise conductive nanoparticles and/or a blend/mixture of two or more different types of polymers, one of said polymers possessing a functional group of the type to which said molecular recognition element is covalently bonded, another of said polymers being conductive to endow the nanofibers with desired electric, ionic and/or photoelectric properties, said conductive polymer being selected from the group consisting of polypyrrole, polythiophene, polyphenol, polyacetelyne, and polyphenylene, and said conductive nanoparticles being selected from the group consisting of titanium dioxide, zinc oxide, tin sulfide and tin oxide.

2-3. (canceled)

4. The membrane assembly as claimed in claim 2 wherein at least some of said electrospun nanofibers comprise a blend/mixture of polyurethane and polyamine.

5. The membrane assembly as claimed in claim 1 wherein at least some of said nanofibers comprise a blend/mixture of two or more different types of polymers, one of said polymers possessing at least one functional group of the type to which a first type of molecular recognition element is covalently bonded, another of said polymers having a second type of functional group, said second type of functional group differing from said first type of functional group and to which a second type of molecular recognition element is covalently bonded.

6-15. (canceled)

16. A membrane assembly adapted for use in capturing an analyte of interest, said membrane assembly comprising:
(a) an electrospun nanofibrous membrane, said electrospun nanofibrous membrane comprising a random mat of electrospun nanofibers, at least some of said electrospun nanofibers including one or more types of functional groups; and
(b) at least one type of molecular recognition element covalently bonded to said random mat via at least one of said types of functional groups, said molecular recognition element being adapted to selectively bind the analyte of interest.

17. The membrane assembly as claimed in claim 16 wherein at least some of said electrospun nanofibers comprise a blend/mixture of two or more types of polymers, at least one of said two or more types of polymers possessing at least one functional group of the type to which the molecular recognition element is covalently bonded.

18. The membrane assembly as claimed in claim 17 wherein said functional group is selected from the group consisting of hydroxyl, aldehyde, carbonyl, carboxyl, amino, amido, thiol, ester, ether, and disulfide groups.

19. The membrane assembly as claimed in claim 16 wherein at least some of said electrospun nanofibers comprise a blend/mixture of two or more different types of polymers, one of said polymers possessing at least one functional group of the type to which a first type of molecular recognition element is covalently bonded, another of said polymers having a second type of functional group, said second type of functional group differing from said first type of functional group and to which a second type of molecular recognition element is covalently bonded.

20. The membrane assembly as claimed in claim 16 wherein at least some of said electrospun nanofibers comprise a blend/mixture of two or more different types of polymers, one of said polymers possessing a functional group of the type to which said molecular recognition element is covalently bonded, another of said polymers being conductive to endow the electrospun nanofibers with desired electric, ionic and/or photoelectric properties.

21. The membrane assembly as claimed in claim 16 wherein at least some of said electrospun nanofibers consist of a single type of polymer possessing at least one functional group of the type to which the molecular recognition element is covalently bonded.

22. The membrane assembly as claimed in claim 21 wherein at least some of said electrospun nanofibers consist of a carboxylated polyvinyl chloride.

23. The membrane assembly as claimed in claim 16 wherein said molecular recognition element is selected from the group consisting of antibodies, aptamers, peptides, peptide nucleic acids and liposaccharides.

24. The membrane assembly as claimed in claim 16 wherein at least some of said electrospun nanofibers include at least one type of molecular recognition element incorporated thereinto.
